(19) United States
(12) Patent Application Publication
(10) Pub. No.: US 2024/0401118 A1
(43) Pub. Date: Dec. 5, 2024

(54) METHODS OF ENHANCING SPATIAL RESOLUTION OF TRANSCRIPTS

(71) Applicant: 10x Genomics, Inc., Pleasanton, CA (US)

(72) Inventors: Augusto Manuel Tentori, Dublin, CA (US); Hanyoup Kim, Foster City, CA (US); Felice Alessio Bava, Rome, CA (US); Rajiv Bharadwaj, Pleasanton, CA (US)

(21) Appl. No.: 18/765,751

(22) Filed: Jul. 8, 2024

Related U.S. Application Data
(62) Division of application No. 17/338,031, filed on Jun. 3, 2021, now Pat. No. 12,031,177.
(60) Provisional application No. 63/034,720, filed on Jun. 4, 2020.

(51) Int. Cl. C12Q 1/6841

(52) U.S. Cl. CPC C12Q 1/6841

(57) ABSTRACT

Provided herein are methods of enhancing spatial resolution of an analyte using sandwich maker system. The methods and systems used herein include a first substrate that includes a plurality of probes that include poly-thymine sequence and a second substrate that includes a plurality of probes comprising a capture domain and a spatial domain.

### CROSS-REFERENCE TO RELATED APPLICATION

This application is a divisional application of U.S. patent application Ser. No. 17/338,031, filed on Jun. 3, 2021, which claims priority to U.S. Provisional Patent Application No. 63/034,720, filed Jun. 4, 2020. The entire contents of the foregoing application are incorporated herein by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Preserving the original spatial distribution of gene expression (also referred to as resolution) is critical for spatial transcriptomics gene expression assays. One option to analyze gene expression is by using multiple substrates. For example, one substrate (e.g., slide) could comprise a barcoded array while another includes a biological specimen (such as a tissue section). Multiple substrate methods utilize random diffusion of the analyte, thus decreasing the resolution on the barcoded substrate. This configuration thus can result in resolution losses due to fundamental mass transport processes. Therefore, there remains a need to increase resolution in spatial methods that incorporate a system that utilizes multiple substrates.

### SUMMARY

Disclosed herein are methods and systems utilizing multiple substrates (e.g., slides) to assess spatial heterogeneity of analytes in a sample. The methods and systems disclosed herein increase the resolution of analyte detection. The methods and systems here utilize multiple substrate arrays, which include at least two slides on either side of a biological sample. Featured herein are methods and systems that include a first slide that includes a plurality of probes. The probes on the first slide are arranged on a lawn across the slide and include a capture domain sequence such as a poly d(T) (e.g., an oligo d(T)) sequence. The second slide which also includes an array of capture probes, and the probes on the second slide include at least a capture domain sequence and spatial barcode. In some instances, the biological sample is provided on the first slide; and after permeabilizing the biological sample, analytes are free to disperse from the biological sample. Because the analytes passively diffuse, they will be captured by the probes on both the first and second slides. In some instances, the methods disclosed herein include capture of the analytes by the probes on the second slide. In doing so, because analytes are captured at random by the probes in the first slide, the area of captured analytes is less diffuse, thereby increasing resolution of the captured analytes.

The disclosure features methods of increasing spatial resolution in an analyte without the use of active migration of an analyte.

Thus, in some instances, this disclosure describes an approach to mitigate resolution losses by including transcript capture functionality, such as the oligo d(T) sequences used in probes of the first slide, where the tissue is placed. During the permeabilization step in the multiple substrate (e.g., slide) configuration, transcripts near the tissue slide surface are captured instead of diffusing to the barcoded bead array, thus reducing spatial broadening. In some instances, this configuration can be applied to barcoded arrays made with printed spots, beads, or microspheres.

Accordingly, in some embodiments, provided herein are methods for determining the abundance and location of an analyte in a biological sample. In some instances, the methods comprise (a) providing a first substrate comprising a plurality of first capture probes, wherein a first capture probe of the plurality of first capture probes comprises a first capture domain, the first substrate comprising a biological sample mounted thereon; (b) providing a second substrate on the opposite side of the first substrate relative to the biological sample, thereby sandwiching the first substrate, the biological sample, and the second substrate, wherein the second substrate comprises a plurality of second capture probes, wherein a second capture probe of the plurality of second capture probes comprises (i) a spatial barcode and (ii) a second capture domain; and (c) hybridizing an analyte to the second capture domain and hybridizing a second analyte to the first capture domain.

Also provided herein are methods for enhancing spatial resolution of an analyte in a biological sample. In some instances, the methods comprise (a) affixing the biological sample to a first substrate comprising a plurality of first capture probes, wherein a first capture probe of the plurality of first capture probes comprises a first capture domain; (b) providing a second substrate on the opposite side of the first substrate relative to the biological sample, thereby creating a sandwich apparatus comprising the first substrate, the biological sample, and the second substrate, wherein the second substrate comprises a plurality of second capture probes, wherein a second capture probe of the plurality of second capture probes comprises (i) a spatial barcode and (ii) a second capture domain; and (c) hybridizing an analyte to the second capture domain and hybridizing a second analyte to the first capture domain, wherein the spatial resolution is enhanced compared to methods for spatial analyte detection (A) which practice one substrate or (B) which practice transfer of an analyte from one substrate comprising a sample to another substrate, wherein the one substrate does not comprise capture probes.

In some instances, the methods disclosed herein further include (d) determining (i) all or a portion of the sequence of the spatial barcode, or a complement thereof, and (ii) all or a portion of the sequence of the one or more analytes captured on the second capture domain, or a complement thereof, and using the sequences of (i) and (ii) to determine the abundance and the location of the analyte in the biological sample.

In some instances, the methods further comprise adding a permeabilization buffer to the biological sample, thereby allowing the analyte and the second analyte to migrate from the biological sample. In some instances, the permeabilization buffer comprises pepsin or proteinase K. In some instances, the permeabilization buffer comprises proteinase K.

In some instances, step (b) of the above methods is performed with the aid of a sample holder comprising: (i) a first member comprising a first retaining mechanism configured to receive the first substrate, (ii) a second member configured to receive the second substrate, and (iii) an alignment mechanism that is connected to at least one of the first member and second member and configured to align the first substrate and the second substrate. In some instances, step (b) of the above methods comprises (i) retaining the first substrate in the first retaining mechanism of the first substrate, (ii) retaining the second substrate in the second retaining mechanism of the second substrate, and (iii) using the alignment mechanism to align the second substrate on the opposite side of the first substrate relative to the biological sample, thereby sandwiching the first substrate, the biological sample, and the second substrate.

In some instances, the first capture domain comprises a poly-thymine (poly(T)) sequence. In some instances, the plurality of second capture probes is arranged on a plurality of beads. In some instances, the second capture probe further comprises one or more functional domains, a unique molecular identifier, a cleavage domain, and combinations thereof.

In some instances, the analyte and the second analyte are RNA molecules. In some instances, the RNA molecules are mRNA molecules.

In some instances, the biological sample is a tissue section sample. In some instances, the biological sample is a fixed sample, a frozen sample, a fresh frozen sample, or a fresh sample. In some instances, the fixed sample is an FFPE biological sample, a PFA fixed sample or an acetone fixed sample.

In some instances, the in situ spatial analysis is performed on the second analyte after the second analyte is captured on the first substrate. In some instances, the in situ spatial analysis comprises detection of the analyte using a detectable moiety, wherein the detectable moiety comprises a nucleic acid sequence that is complementary to the second analyte or a complement thereof, and/or the second probe or a complement thereof. In some instances, the detectable moiety is a fluorescent probe. In some instances, the methods on the first substrate further include in situ amplification of the second analyte or a complement thereof.

In some instances, the determining step comprises sequencing, wherein the sequencing is selected from in situ sequencing, Sanger sequencing methods, next-generation sequencing methods, and nanopore sequencing.

Also provided herein are kits and systems. In some instances, the kits include (a) a first substrate comprising a plurality of first capture probes, wherein a first capture probe of the plurality of first capture probes comprises a first capture domain; (b) a second substrate comprising a plurality of second capture probes, wherein a second capture probe of the plurality of second capture probes comprises (i) a spatial barcode and (ii) a second capture domain; and (c) instructions for performing any of the methods provided herein. In some instances, the systems provided herein are for determining the abundance and location of an analyte in a biological sample. In some instances, the systems comprise (a) a first substrate comprising a plurality of first capture probes, wherein a first capture probe of the plurality of first capture probes comprises a first capture domain; (b) a second substrate comprising a plurality of second capture probes, wherein a second capture probe of the plurality of second capture probes comprises (i) a spatial barcode and (ii) a second capture domain; (c) a biological sample holder comprising: (i) a first member comprising a first retaining mechanism configured to receive the first substrate, (ii) a second member configured to receive the second substrate, and (iii) an alignment mechanism that is connected to at least one of the first member and second member and configured to align the first substrate and the second substrate; and (d) the biological sample.

In some embodiments, also provided herein are methods for enhancing spatial resolution of one or more analytes in a biological sample, the method comprising: (a) affixing the biological sample to a first slide comprising a plurality of first capture probes, wherein a first capture probe of the plurality of first capture probes comprises a first capture domain; (b) providing a second slide in opposition and superior to the first slide, wherein the second slide comprises a plurality of second capture probes, wherein a second capture probe of the plurality of second capture probes comprises (i) a spatial barcode and (ii) a second capture domain; (c) providing a permeabilization buffer between the biological sample and the second slide, thereby allowing the one or more analytes to migrate from the biological sample; and (d) determining (i) all or a portion of the sequence of the spatial barcode, or a complement thereof, and (ii) all or a portion of the sequence of the one or more analytes, or a complement thereof, and using the sequences of (i) and (ii) to determine the location of the one or more analytes in the biological sample; wherein the resolution is enhanced compared to methods for spatial analyte detection which practice one spatial array slide.

In some embodiments, the first capture domain comprises a poly-thymine sequence.

In some embodiments, the first capture probes are distributed on the first slide inferior to the biological sample.

In some embodiments, the first slide is printed with first capture probes.

In some embodiments, a distance between the biological sample and the first capture probes is about 0.5 μm, about 1.0 μm, about 1.5 μm, about 2.0 μm, about 2.5 μm, about 3.0 μm, about 3.5 μm, about 4.0 μm, about 4.5 μm, about 5.0 μm, or more.

In some embodiments, at least about 80%, or at least about 90% of the one or more analytes are hybridized to one or more of the second capture probes.

In some embodiments, the second capture domain comprises a poly-thymine sequence.

In some embodiments, the second capture probes are arranged on a plurality of beads.

In some embodiments, a bead in the plurality of beads has a diameter of about 5 μm, about 6 μm, about 7 μm, about 8 μm, about 9 μm, or about 10 μm.

In some embodiments, the second capture probe further comprises one or more cleavage domains, a functional domain, and a unique molecular identifier.

In some embodiments, the functional domain is a primer sequence.

In some embodiments, the one or more analytes is RNA.

In some embodiments, the RNA is mRNA.

In some embodiments, the biological sample is a tissue sample.

In some embodiments, the biological sample is a fixed sample, a frozen sample, or a fresh sample.

In some embodiments, the biological sample is an FFPE biological sample, a PFA fixed sample or an acetone fixed sample.

In some embodiments, a step of fixing the biological sample.

In some embodiments, the permeabilization buffer releases the one or more analytes from the biological sample.

In some embodiments, the permeabilization buffer comprises proteinase K, pepsin, collagenase, a detergent, one or more ribonuclease inhibitor, or combinations thereof.

In some embodiments, the detergent is selected from sodium dodecyl sulfate (SDS), polyethylene glycol tert-octylphenyl ether, polysorbate 80, polysorbate 20, or combinations thereof.

In some embodiments, the permeabilization buffer comprises a hydrogel.

In some embodiments, the hydrogel comprises one or more of dried reagents or monomers to deliver permeabilization reagents when the hydrogel is applied to a biological sample.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce spatially-barcoded cells or cellular contents.

FIG. 7 shows a schematic example of the spatial assay disclosed herein.

FIG. 8A shows a schematic example of a spatial assay without capture probes on the first substrate.

FIG. 8B shows a schematic example of a spatial assay with capture probes on the first substrate.

FIG. 9 shows a schematic example of the spatial assay with first capture probes on a first substrate and second capture probes on a second substrate.

FIG. 10 shows a schematic example of the spatial assay with first capture probes on a first substrate and second capture probes on a second substrate. In this embodiment, a fluorescent image is obtained by detecting analytes that hybridize to the probes on the first substrate and gene expression analysis is performed using the analytes that hybridize to the probes on the second substrate.

FIG. 11 depicts results from an experiment comparing a non-sandwich control and a sandwich configuration permeabilization condition.

FIG. 12 depicts a comparison between a non-sandwich control and a sandwich configuration permeabilization condition.

FIG. 13 depicts a spatial clustering analysis and analysis of hippocampal transcript Hpca, comparing non-sandwich control and sandwich configuration permeabilization conditions.

### DETAILED DESCRIPTION

### I. Introduction

Disclosed herein are methods, kits, systems, and apparatuses for detecting one or more analytes in a biological sample. In particular, disclosed herein are methods, kits, systems, and apparatuses that utilize two substrates comprising capture probes. Disclosed herein are methods that increase the resolution of an analyte captured on a spatial array using sandwiching methods. In a traditional sandwich method, analytes migrating from the biological sample to a spatial array have the capability of migrating laterally (e.g., along the transverse section of a biological sample). Laterally migrating (or diffusing) analytes tend to be captured by probes that are not most proximate to the origin of the analyte in the biological sample. Thus, detection of the captured analytes would be more diffuse on the array compared to their actual location in the biological sample, leading to a less accurate detection of analytes in a sandwich method. To address this, the present disclosure provides a sandwich method in which analytes are captured on both substrates of a sandwiching apparatus. Indiscriminately capturing analytes on the substrate upon which the biological sample is placed decreases the overall lateral diffusion of analytes in a biological sample, and leads to more accurate detection of analytes on the second substrate in the sandwiching apparatus. Thus, provided herein are methods kits, systems, and apparatuses for increasing resolution of detection of analytes on a spatial array.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e g, mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10x Genomics Support Documentation website, and can be used herein in any combination. Further non-limiting aspects of spatial analysis methodologies and compositions are described herein.

Some general terminologies that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

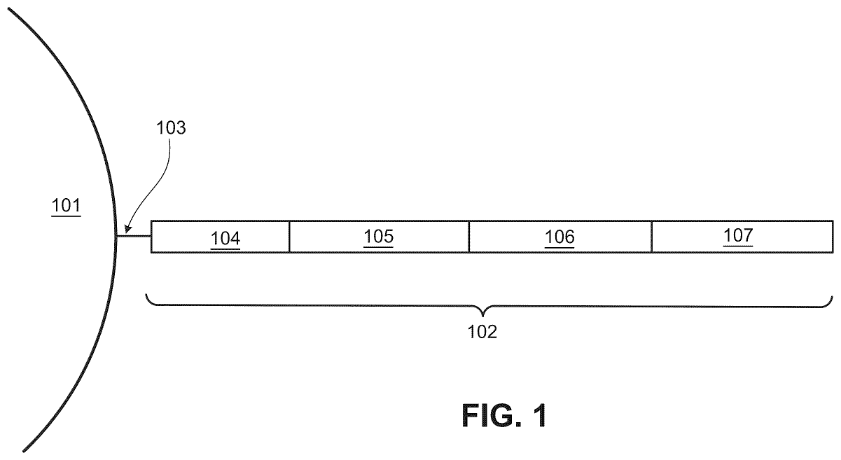

FIG. 1 is a schematic diagram showing an exemplary capture probe, as described herein. As shown, the capture probe 102 is optionally coupled to a feature 101 by a cleavage domain 103, such as a disulfide linker. The capture probe can include afunctional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

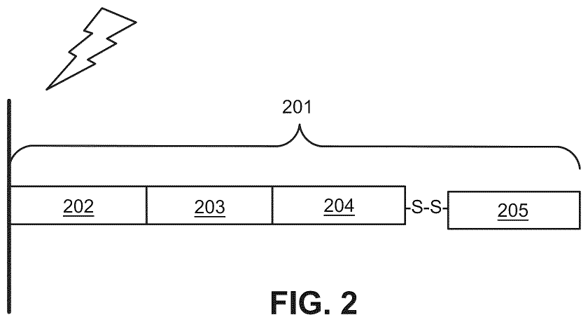

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain

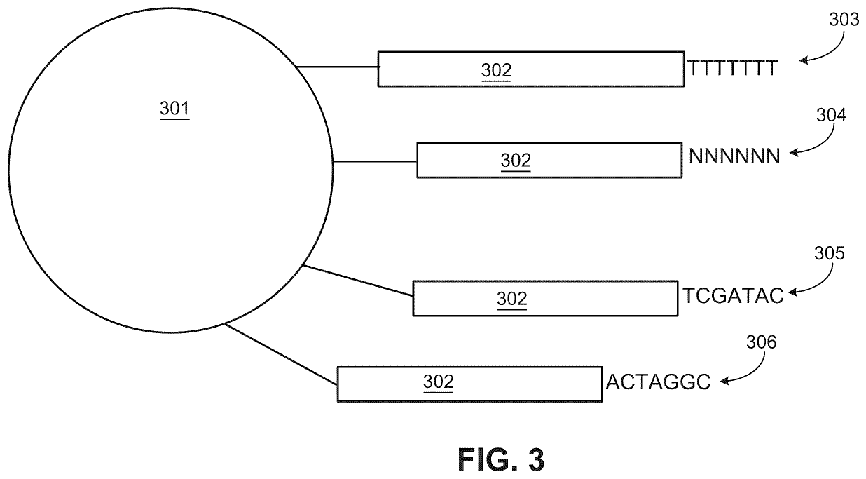

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II) (b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

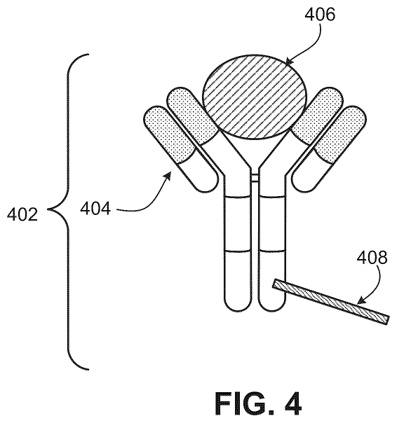

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

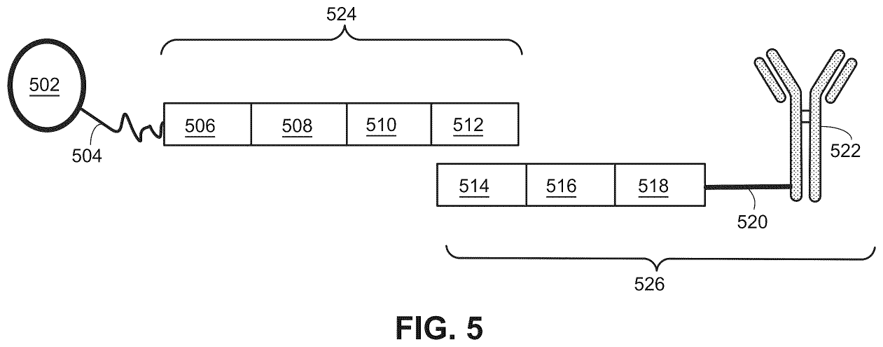

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

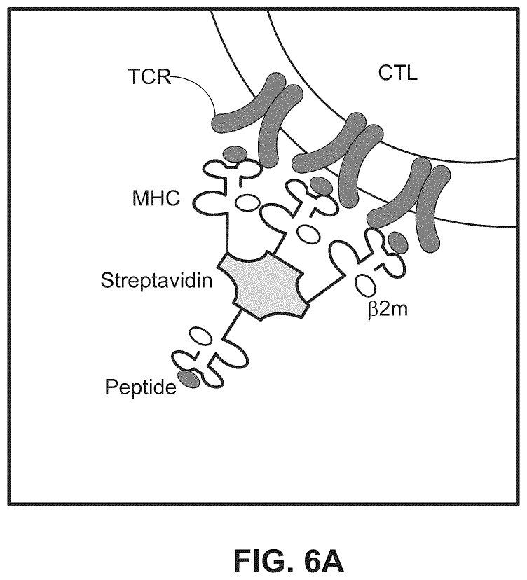

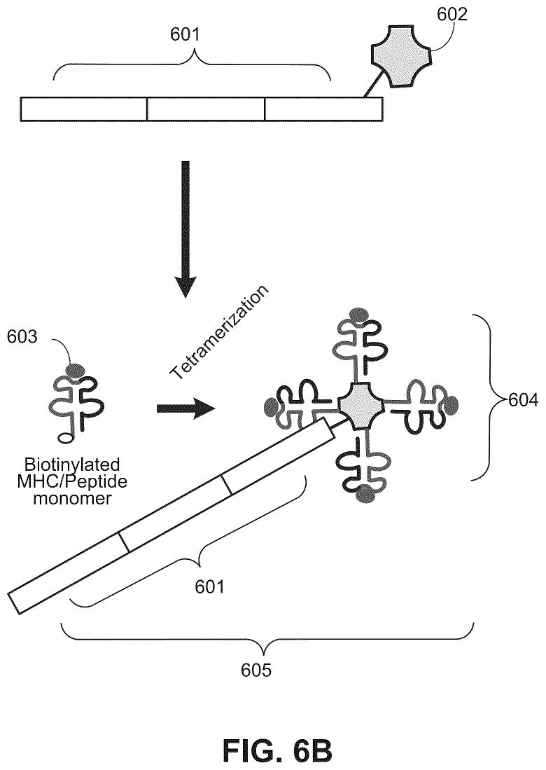

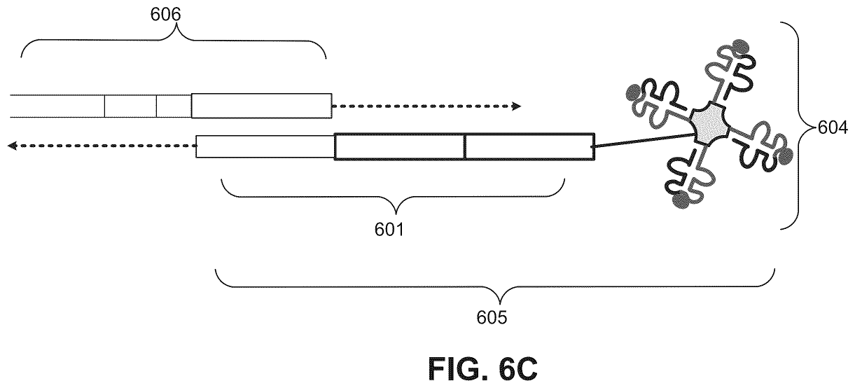

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MHC/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of the corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction). Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663. Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease. In some embodiments, the endonuclease is an RNAse. In some embodiments, the endonuclease is one of RNase A, RNase C, RNase H, and RNase I. In some embodiments, the endonuclease is RNAse H. In some embodiments, the RNase H is RNase H1 or RNase H2. The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev D, dated October 2020), and/or the Visium Spatial Tissue Optimization Reagent Kits User Guide (e.g., Rev D, dated October 2020). In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Enhancing Spatial Resolution on a Spatial Array

### (a) Introduction

Disclosed herein are methods, kits, systems, and apparatuses utilizing a spatial array system to assess spatial heterogeneity of analytes in a sample. The methods and systems disclosed herein increase the resolution of heterogeneity detection of one or more analytes on the second sample. The methods and systems here utilize spatial arrays, which include at least two substrates (e.g., slides) on opposite sides of a biological sample.

Featured herein are methods for determining the abundance and location of an analyte in a biological sample. In some instances, the methods include (a) providing a first substrate comprising a plurality of first capture probes, wherein a first capture probe of the plurality of first capture probes comprises a first capture domain, the first substrate comprising a biological sample mounted thereon; (b) providing a second substrate on the opposite side of the first substrate relative to the biological sample, thereby sandwiching the first substrate, the biological sample, and the second substrate, wherein the second substrate comprises a plurality of second capture probes, wherein a second capture probe of the plurality of second capture probes comprises (i) a spatial barcode and (ii) a second capture domain; and (c) hybridizing an analyte to the second capture domain and hybridizing a second analyte to the first capture domain

Also featured herein are methods for enhancing spatial resolution of an analyte in a biological sample. In some instances, the methods include (a) affixing the biological sample to a first substrate comprising a plurality of first capture probes, wherein a first capture probe of the plurality of first capture probes comprises a first capture domain; (b) providing a second substrate on the opposite side of the first substrate relative to the biological sample, thereby creating a sandwich apparatus comprising the first sample, the biological sample, and the second sample, wherein the second substrate comprises a plurality of second capture probes, wherein a second capture probe of the plurality of second capture probes comprises (i) a spatial barcode and (ii) a second capture domain; and (c) hybridizing an analyte to the second capture domain and hybridizing a second analyte to the first capture domain, wherein the spatial resolution is enhanced compared to methods for spatial analyte detection (A) which practice one substrate or (B) which practice transfer of an analyte from one substrate comprising a sample to another substrate, wherein the one substrate does not comprise capture probes.

In some instances, the methods further include determining (i) all or a portion of the sequence of the spatial barcode, or a complement thereof, and (ii) all or a portion of the sequence of the one or more analytes captured on the second capture domain, or a complement thereof, and using the sequences of (i) and (ii) to determine the abundance and the location of the analyte in the biological sample.

Also featured herein are methods and systems that include a first substrate that includes a plurality of probes. The probes on the first substrate are arranged on a lawn across the substrate or an area of the substrate and include a capture domain sequence such as a poly(T) (e.g., a poly-thymine sequence or an oligo d(T)) sequence). The second substrate also includes a lawn across the substrate or an area of the substrate of capture probes, and the probes on the second substrate include at least a capture domain sequence and spatial barcode. In some instances, the biological sample is provided on the first substrate; after permeabilizing the biological sample analytes are free to disperse from the biological sample. As the analytes passively diffuse, they can be captured by the probes both on the first substrate and the second substrate or on areas of substrates. In some instances, the methods disclosed herein include capture of the analytes by the probes on the second substrate. As some analytes in close proximity to the first substrate are captured by the probes on the first substrate, the area of captured analytes on the second substrate can be less diffuse, thereby increasing resolution of the captured analytes.

Thus, in some instances, this disclosure describes an approach to mitigate resolution losses by including transcript capture functionality, such as the oligo d(T) sequences used in probes of the first substrate, where the tissue is placed. During the permeabilization step in the assay configuration, transcripts in proximity to the tissue slide surface are captured instead of diffusing to the capture probes on the second substrate, wherein the second substrate may comprise a barcoded array, e.g., a barcoded bead array (e.g., an array of beads wherein the capture probes comprising a capture domain and a spatial barcode are located on the beads), thus reducing spatial broadening. In some instances, this configuration can be applied to barcoded arrays made with printed spots, beads, or microspheres.

The methods disclosed herein enhance resolution of the location of a transcript by increasing the spatial resolution of analytes of the biological sample. In some instances, the methods include affixing the biological sample to a substrate comprising a plurality of first capture probes, wherein a first capture probe of the plurality of first capture probes comprises a first capture domain; providing an array comprising a plurality of second capture probes, wherein a second capture probe of the plurality of second capture probes comprises (i) a spatial barcode and (ii) a second capture domain; providing one or more permeabilization materials; wherein the one or more permeabilization materials are disposed between the biological sample and the array; and determining (i) all or a portion of the sequence of the spatial barcode, or a complement thereof, and (ii) all or a portion of the sequence of the first analyte, or a complement thereof, and using the sequences of (i) and (ii) to determine the location of the first analyte in the biological sample; wherein the resolution is enhanced compared to methods wherein the sample substrate does not comprise the plurality of first capture probes or methods that for spatial analyte detection which practice one spatial array substrate.

### (b) Methods and Systems of Enhancing Spatial Resolution

The following section provide guidance on the steps of performing the methods to enhance spatial resolution and to detect an analyte in a biological sample using the sandwich configuration disclosed herein. Additional methods of spatial analysis are found in WO 2020/176788, WO 2020/123320, and U.S. Patent Application Publication No. 2020/0277663, each of which is incorporated by reference in its entirety.

### (i) Providing the Substrates and Generating the Sandwich Configuration

In some instances, a first substrate is provided. As disclosed herein, the first substrate can be a glass slide. In some instances, a biological sample is placed on the first substrate.

In some instances, the biological sample is provided on a first substrate. As used herein, the first substrate is a substrate that allows the biological sample to adhere to the substrate. In some instances, the first substrate is a glass substrate. In some instances, the first substrate includes materials formed from various glasses, substrate s formed from various polymers, hydrogels, layers and/or films, membranes (e.g., porous membranes), flow cells, wafers, plates, or combinations thereof. In some instances, the first substrate includes a plurality of probes adhered to the substrate. In some instances, one or more probes in the plurality includes a capture domain that is complementary to a sequence on an analyte. In some instances, the capture domain includes a poly-thymine (e.g., also called poly(T), poly-d(T), or oligo d(T) throughout) sequence that is complementary to a poly-adenylation sequence. In some instances, the capture probes are distributed on the first substrate inferior to the biological sample. In some instances, a probe in the plurality is about 20, about 25, about 30, about 35, about 40, about 45, about 50, about 55, about 60, about 65, about 70, about 75, about 80, about 85, about 90, about 95, about 100, or more single-stranded nucleotides in length. In some instances, a probe includes one or more non-naturally occurring nucleotides. In some instances, the probes are distributed equally on the lawn of the substrate. In some instances, the probes on the first substrate are adhered directly (e.g., as described herein). In some instances, the probes are placed using printed spots (e.g., as described herein).

In some instances, a second substrate is placed superior to the biological sample opposite to the first substrate, creating a sandwich configuration wherein the biological sample and the permeabilization buffer are located in between the two substrates. In some instances, the second substrate is placed below the biological sample, opposite to the first substrate creating a sandwich configuration wherein the biological sample and the permeabilization buffer are located in between the two substrates. In some instances, the second substrate is a glass substrate. In some instances, the second substrate includes various glasses, substrates formed from various polymers, hydrogels, layers and/or films, membranes (e.g., porous membranes), flow cells, wafers, plates, or combinations thereof. In some instances, the second substrate includes a plurality of probes (e.g., as described herein). In some instances, one or more probes in the plurality includes a spatial barcode and a capture domain In some instances, a probe on the second substrate includes a capture domain (e.g., a poly(T) sequence), a unique molecular identifier, a functional sequence such as a primer, a spatial barcode, or combinations thereof. For example, a probe on the second substrate can be a probe as described in FIG. 1. In some instances, the probes on the second substrate are adhered directly to the substrate (e.g., as described herein). In some instances, the probes on the second substrate are placed using printed spots (e.g., as described herein).

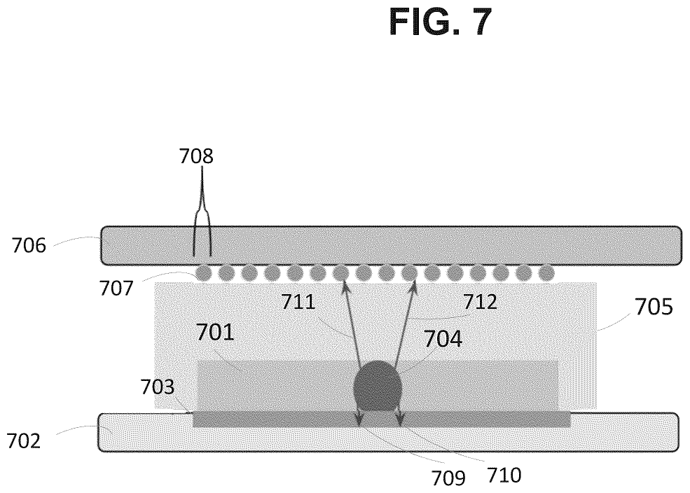

In some instances, the system includes a biological sample sandwiched between two substrates. Referring to FIG. 7, a biological sample 701 comprising one or more cells (704 represents one cell) is placed on a first substrate 702. The first substrate 702 includes a lawn 703 of probes that include a poly-thymine sequence. The lawn (i.e., plurality) of probes can be associated with a whole substrate, parts of a substrate, or defined regions on a substrate. The system includes a permeabilization buffer 705 that is added to the biological sample 701, allowing analytes to diffuse from the cell 704. Analytes diffuse from the cell passively in any direction, for example, vertically, horizontally, or laterally, (e.g., arrows represented as 709, 710, 711, and 712). In some instances, analytes diffuse from the cell to the lawn 703 of probes on the first substrate. There, they are captured by a probe in the lawn (e.g., in the plurality of probes). Analytes also migrate to a second substrate 706, which includes a second array (i.e., plurality) of probes. A probe or bead 707 (which comprises probes) in the second array includes a capture domain sequence (e.g., a poly-thymine sequence) and a spatial barcode. In some instances, the analyte migrates to the second substrate in a vector, for example, as shown by 711 or 712. After migrating to the second substrate, the analyte can be extended, amplified, and sequence using methods disclosed herein.

In some instances, still referring to FIG. 7, the first substrate 702 and the second substrate 706 are sandwiched together and form substantially parallel planes. In some instances, the angle of migration of the analyte is measured as an angle from the first substrate 702. In some instances, the angle 711 is 90 degrees relative to the first substrate 702. In some instances, the angle is about 85, about 80, about 75, about 70, about 65, about 60, about 55, about 50, or about 45 degrees relative to the first substrate 702.

In some embodiments, the first substrate 702 and the second substrate 706 are sandwiched together in a sandwiching process. As shown in FIG. 7, during the exemplary sandwiching process, the first substrate is aligned with the second substrate, such that at least a portion of the biological sample is aligned with at least a portion of the array (e.g., aligned in a sandwich configuration). In some embodiments, the first substrate is aligned with the second substrate such that at least a portion of the biological sample is vertically aligned with at least a portion of the array. As shown, the second substrate is in a superior position to the first substrate. In some embodiments, the first substrate may be positioned superior to the second substrate. In some embodiments, the first and second substrates are aligned to maintain a gap or separation distance between the two substrates. When the first and second substrates are aligned, one or more analytes are released from the biological sample and actively or passively migrate to the array for capture. In some embodiments, the migration occurs while the aligned portions of the biological sample and the array are contacted with a reagent medium (e.g., permeabilization buffer) 705. The released one or more analytes may actively or passively migrate across the gap via the reagent medium 705 toward the capture probes or beads 707 on second substrate 706, and be captured by the capture probes 707.

In some embodiments, the separation distance between first and second substrates is maintained between 2 microns and 1 mm (e.g., between 2 microns and 800 microns, between 2 microns and 700 microns, between 2 microns and 600 microns, between 2 microns and 500 microns, between 2 microns and 400 microns, between 2 microns and 300 microns, between 2 microns and 200 microns, between 2 microns and 100 microns, between 2 microns and 25 microns, between 2 microns and 10 microns), measured in a direction orthogonal to the surface of first substrate that supports sample. In some instances, the distance is 2 microns. In some instances, the distance is 2.5 microns. In some instances, the distance is about 2, 3, 4, 5, 6, 7, 8, 9, 10, 11, 12, 13, 14, 15, 16, 17, 18, 19, 20, 21, 22, 23, 24, or 25 microns. In some embodiments, second substrate is placed in direct contact with the sample on the first substrate ensuring no diffusive spatial resolution losses. In some embodiments, the separation distance is measured in a direction orthogonal to a surface of the first substrate that supports the biological sample.

In some embodiments, the first and second substrates are placed in a substrate holder (e.g., an array alignment device) configured to align the biological sample and the array. In some embodiments, the device comprises a sample holder. In some embodiments, the sample holder includes first and second members that comprise first and second retaining mechanisms configured to retain the first and second substrates, respectively. The device can include an alignment mechanism that is connected to at least one of the members and aligns the first and second members. Thus, the devices of the disclosure can advantageously align the first substrate and the second substrate and any samples, barcoded probes, or permeabilization reagents that may be on the surface of the first and second substrates.

In some instances, a sample holder is provided as part of the sandwiching mechanism (e.g., sandwiching apparatus) used in the methods disclosed herein. In some instances, the sample holder includes a first member that includes a first retaining mechanism that retains substrate with sample. In some instances, the sample holder also includes a second member that includes a second retaining mechanism that retains second substrate with a feature array. An alignment mechanism is connected to at least one of first and second members or to both first and second members. During an alignment and contacting procedure, an alignment mechanism functions to align the first and second members, thereby ensuring that sample and feature array are also aligned and brought into contact to facilitate analysis of sample.

In some embodiments, the alignment mechanism can be implemented as a rotating actuator connected to the first and second members. One example of such a rotating actuator is a hinge. In some instances, once a substrate-mounted sample is positioned in the first member and a substrate-mounted feature array is positioned in the second member, rotation of one of the members about the hinge axis aligns members and, and also aligns sample and feature array. The members can be rotated about the hinge axis until the sample and feature array are aligned and in contact. In some instances, the rotating actuator is implemented as a folding member Folding member can be formed from a variety of materials, including compliant materials such as rubber and vinyl, metals and metal alloys, and plastics.

In certain embodiments, the rotating actuator can include at least one arm. In some instances, the rotating actuator can include multiple arms (e.g., 2 or more, 3 or more, 4 or more, or even more). In some instances, sample holder is implemented as a unitary (i.e., one-piece) device. In some instances, Sample holder can also be implemented as a two-piece device, with first and second members being separate but reproducibly connectable via the alignment mechanism. When the first and second members are brought into proximity, connectors engage with receivers, aligning first and second members, and also aligning sample with the feature array. It should be noted that while connectors are positioned on second member and receivers are positioned on first member, the reverse could also be true. Moreover, first and second members could each have one or more connectors and one or more receivers.

The first retaining mechanism can be implemented in various ways. In some embodiments, first retaining mechanism can correspond to a recess dimensioned to receive first substrate. Further, a gasket can optionally be positioned within the recess to maintain an interference fit between the edges of the recess and first substrate. In certain embodiments, first retaining mechanism can correspond to one or more members positioned to apply a force to first substrate, in particular, to maintain contact between first substrate and first member. Examples of such members include, but are not limited to, clips, screws and other threaded retaining fasteners, and members that snap-fasten or otherwise engage with first member. The members can apply a force to the sample bearing surface of first substrate and/or to one or more lateral surfaces first substrate.

In general, second retaining mechanism can correspond to any of the different types of retaining mechanisms discussed above in connection with first retaining mechanism. First and second retaining mechanisms and can be different or the same.

In some embodiments, the first member includes a first aperture. The first aperture can be positioned, for example, so that when the first substrate is retained in first member, first aperture is aligned with a sample region (e.g., a region where sample is typically located, or which is designated for placement of sample) on first substrate. Aperture can be positioned so that sample can be viewed from the back surface of first member (i.e., the surface opposite to the surface that supports first substrate) through first aperture, and one or more images of sample can be obtained through first aperture.

As described above, a reagent medium can be positioned on a first or second substrate. More generally, however, the first or second substrate may further comprise a reagent medium placed thereon. In certain embodiments, the reagent medium includes a permeabilization reagent (e.g., a solid, liquid, gel, or dried permeabilization reagent). In some embodiments, the reagent medium includes one or more additional components. For example, the additional components can include a hydrogel compound or layer with an embedded permeabilization reagent. In some embodiments, the second member includes at least one aperture. More generally, the second member can include one or more (e.g., 2 or more, 3 or more, 4 or more, 5 or more, 6 or more, 8 or more, 10 or more, 15 or more, 20 or more, 30 or more, 40 or more, 50 or more, or even more) second apertures. In certain embodiments, the second aperture is aligned with at least a portion of the sample region on substrate when the first and second members and are aligned. A second aperture can used for various purposes. In some embodiments, for example, the feature array and/or the sample can be viewed or imaged through second aperture. Viewing/imaging can be used to adjust the relative positions of the feature array and the sample to improve alignment, for example.

In certain embodiments, one or more bounding surfaces of the second aperture and a back surface of the second substrate (i.e., a surface of the second substrate that is opposite to the surface of the second substrate that faces the sample and that supports feature array) cooperate to form a reagent well. A reagent solution (e.g., comprising a permeabilization reagent) added to the reagent well is contained by the bounding surfaces of the second aperture. If the second substrate is formed from a permeable or semi-permeable material, the reagent solution can permeate (e.g., by diffusion) through the back surface of the second substrate and contact the sample.

In some embodiments, the sample holder includes a first adjustment mechanism connected to the first member. The first adjustment mechanism translates the first substrate in at least one direction parallel to the surface of the first substrate that supports the sample. In some embodiments, the first adjustment mechanism translates the first substrate in two directions parallel to the surface of the first substrate.

The first adjustment mechanism can be implemented in various ways. In some embodiments, for example, the first adjustment mechanism includes one or more thumbscrews or linear actuators that can be used to translate the first substrate.

In addition to aligning the first and second members, the alignment mechanism is also configured to maintain a separation between the first and second substrates (and the first and second members) when the substrates (and members) are aligned. For example, the separation can be maintained such that at least a portion of the sample contacts the reagent medium (e.g., the feature array of the reagent medium).

In certain embodiments, the alignment mechanism maintains the first and second substrates in an approximately parallel relationship when the substrates (and the first and second members) are aligned. An included angle between the first and second substrates in such circumstances can be 2 degrees or less (e.g., 1 degree or less, 0.5 degrees or less, 0.25 degrees or less).

In some embodiments, the sample holder can include one or more spacing members that assist in maintaining the spacing and/or approximately parallel arrangement of the first and second substrates. Spacing members can be connected to either or both of the first and second members.

In certain embodiments, the sample holder includes a second adjustment mechanism. The second adjustment mechanism adjusts a distance of the separation between the first and second substrates (i.e., in a direction orthogonal to the surface of the first substrate that supports the sample). In certain embodiments, the adjustment mechanism is connected to both members.

The second adjustment mechanism can be implemented in various ways. In some embodiments, the second adjustment mechanism includes one or more thumbscrews or adjustable pins or posts. In certain embodiments, the second adjustment mechanism includes one or more linear actuators. In some embodiments, the second adjustment mechanism includes a swellable or expandable membrane, gasket, or layer positioned between the first and second members.

As a subsequent step in an analytical workflow, after the sample and the feature array have been brought into contact by the sample holder, the sample holder can be introduced into a thermocycler to promote capture of analytes from the sample by the feature array. The sample holder can be inserted directly into a suitable thermocycler for this purpose. Alternatively, in some embodiments, the sample holder can be coupled to a thermocycler adapter and the coupled holder and adapter inserted into a thermocycler. Exemplary devices and exemplary sample holders are described in PCT Patent Application Publication No. WO 2020/123320, which is incorporated by reference in its entirety.

In some instances, the probes on the first and/or second substrate are adhered to beads (e.g., as described herein). In some instances, the probes are placed on the first and/or second substrate using microspheres (e.g., as described herein). In some instances, the beads or microspheres that include probes are associated with, or affixed to, the first and/or second substrate. For example, in some instances capture probe containing beads or microsphere are affixed directly or indirectly to a substrate via surface chemistries, hydrogel, and the like.

In some instances, the diameter 708 of a bead the is adhered to the probe on the second substrate is about 1 μm, about 2 μm, about 3 μm, about 4 μm, about 5 μm, about 6 μm, about 7 μm, about 8 μm, about 9 μm, about 10 μm, about 15 μm, about 20 μm, or more.

The substrate holder is compatible with a variety of different schemes for contacting the aligned portions of the biological sample and array with the reagent medium to promote analyte capture. In some embodiments, the reagent medium is deposited directly on the second substrate (e.g., forming a reagent medium that includes the permeabilization reagent and the feature array), and/or directly on the first substrate. In some embodiments, the reagent medium is deposited on the first and/or second substrate, and then the first and second substrates aligned in the sandwich configuration such that the reagent medium contacts the aligned portions of the biological sample and array. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some instances, the aligned portions of the biological sample and the array are in contact with the reagent medium for about 1 minute. In some instances, the aligned portions of the biological sample and the array are in contact with the reagent medium for about 5 minutes. In some instances, the aligned portions of the biological sample and the array are in contact with the reagent medium in the gap for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes. In some instances, the aligned portions of the biological sample and the array are in contact with the reagent medium for about 30 minutes.

In some embodiments, following initial contact between sample and a permeabilization agent, the permeabilization agent can be removed from contact with sample (e.g., by opening substrate holder). In some embodiments, the substrate holder is opened before complete permeabilization of sample. For example, in some embodiments, only a portion of sample is permeabilized, and only a portion of the analytes in sample may be captured by feature array. In some instances, the reduced amount of analyte captured and available for detection can be offset by the reduction in lateral diffusion that results from incomplete permeabilization of sample. In general, the spatial resolution of the assay is determined by the extent of analyte diffusion in the transverse direction (i.e., orthogonal to the normal direction to the surface of sample). The larger the distance between the sample on the first substrate and the feature array on the second substrate, the greater the extent of diffusion in the transverse direction, and the concomitant loss of resolution. Analytes liberated from a portion of the sample closest to the feature array have a shorter diffusion path, and therefore do not diffuse as far laterally as analytes from portions of the sample farthest from the feature array. As a result, in some instances, incomplete permeabilization of the sample (by reducing the contact interval between the permeabilization agent and the sample) can be used to further increase spatial resolution in the assay. In some embodiments, the substrate holder is opened following complete or substantially complete permeabilization of the sample.

In some instances, the device is configured to control a temperature of the first and second substrates. In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In an exemplary embodiment, the second substrate is contacted with the permeabilization reagent. In some embodiments, the permeabilization reagent is dried. In some embodiments, the permeabilization reagent is a gel or a liquid. Also in the exemplary embodiment, the biological sample is contacted with buffer. Both the first and second substrates are placed at lower temperature to slow down diffusion and permeabilization efficiency. Alternatively, in some embodiments, the sample can be contacted directly with a liquid permeabilization reagent without inducing an unwanted initiation of permeabilization due to the substrates being at the second temperature. In some embodiments, the low temperature slows down or prevents the initiation of permeabilization. In a second step, keeping the sample holder and substrates at a cold temperature (e.g., at the first or second temperatures) continues to slow down or prevent the permeabilization of the sample. In a third step, the sample holder (and consequently the first and second substrates) is heated up to initiate permeabilization. In some embodiments, the sample holder is heated up to a third temperature. In some embodiments, the third temperature is above room temperature (e.g., 25 degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, analytes that are released from the permeabilized tissue of the sample diffuse to the surface of the second substrate and are captured on the array (e.g., barcoded probes) of the second substrate. In a fourth step, the first substrate and the second substrate are separated (e.g., pulled apart) and temperature control is stopped.

In some embodiments, where either the first substrate or substrate second (or both) includes wells, a permeabilization solution can be introduced into some or all of the wells, and then the sample and the feature array can be contacted by closing the sample holder to permeabilize the sample. In certain embodiments, a permeabilization solution can be soaked into a hydrogel film that is applied directly to the sample, and/or soaked into features (e.g., beads) of the array. When the first and second substrates are aligned in the sandwich configuration, the permeabilization solution promotes migration of analytes from the sample to the array.

In certain embodiments, different permeabilization agents or different concentrations of permeabilization agents can be infused into array features (e.g., beads) or into a hydrogel layer as described above. By locally varying the nature of the permeabilization reagent(s), the process of analyte capture from the sample can be spatially adjusted.

In some instances, migration of the analyte from the biological sample to the second substrate is passive (e.g., via diffusion). Alternatively, in certain embodiments, migration of the analyte from the biological sample is performed actively (e.g., electrophoretic, by applying an electric field to promote migration). In some instances, first and second substrates can include a conductive epoxy. Electrical wires from a power supply can connect to the conductive epoxy, thereby allowing a user to apply a current and generate an electric field between the first and second substrates. In some embodiments, electrophoretic migration results in higher analyte capture efficiency and better spatial fidelity of captured analytes (e.g., on a feature array) than random diffusion onto matched substrates without the application of an electric field (e.g., via manual alignment of the two substrates). Exemplary methods of electrophoretic migration, are described in WO 2020/176788, which is hereby incorporated by reference in its entirety.

Loss of spatial resolution can occur when analytes migrate from the sample to the feature array and a component of diffusive migration occurs in the transverse (e.g., lateral) direction, approximately parallel to the surface of the first substrate on which the sample is mounted. To address this loss of resolution, in some embodiments, a permeabilization agent deposited on or infused into a material with anisotropic diffusion can be applied to the sample or to the feature array. The first and second substrates are aligned by the sample holder and brought into contact. A permeabilization layer that includes a permeabilization solution infused into an anisotropic material is positioned on the second substrate.

In some embodiments, the feature array can be constructed atop a hydrogel layer infused with a permeabilization agent. The hydrogel layer can be mounted on the second substrate, or alternatively, the hydrogel layer itself may function as the second substrate. When the first and second substrates are aligned, the permeabilization agent diffuses out of the hydrogel layer and through or around the feature array to reach the sample. Analytes from the sample migrate to the feature array. Direct contact between the feature array and the sample helps to reduce lateral diffusion of the analytes, mitigating spatial resolution loss that would occur if the diffusive path of the analytes was longer.

In some embodiments, the workflow includes provision of the first substrate comprising the biological sample. In some embodiments, the workflow includes, mounting the biological sample onto the first substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

In some embodiments, the biological sample on the first substrate is stained using any of the methods described herein. In some instances, the biological sample is imaged, capturing the stain pattern created during the stain step. In some instances, the biological sample then is destained prior to the sandwiching process.

In some instances, after the sandwiching process the first substrate and the second substrate are separated (e.g., such that they are no longer aligned in a sandwich configuration, also referred to herein as opening the sandwich). In some embodiments, subsequent analysis (e.g., reverse transcription, cDNA synthesis, library preparation, and sequences) can be performed on the captured analytes after the first substrate and the second substrate are separated.

In some embodiments, the process of transferring an analyte from the first substrate to the second substrate is referred to interchangeably herein as a “sandwich process,” “sandwiching process,” or “sandwiching”. The sandwich process is further described in PCT Patent Application Publication No. WO 2020/123320, which is incorporated by reference in its entirety.

In some instances, referring to FIG. 7, the permeabilization buffer 705 includes proteinase K, pepsin, collagenase, a detergent, one or more ribonuclease inhibitor, or combinations thereof. In some instances, the detergent is selected from sodium dodecyl sulfate (SDS), polyethylene glycol tert-octylphenyl ether, polysorbate 80, polysorbate 20, N-lauroylsarcosine, or combinations thereof. In some instances, the permeabilization buffer 705 comprises a hydrogel.

Prior to analyte capture, in some instances, biological samples can be stained using a wide variety of stains and staining techniques. In some instances, the biological sample is a tissue section on a substrate (e.g., a slide; e.g., a 10 μm biological section section). In some instances, the tissue section is about 5, 6, 7, 8, 9, 10, 11, 12, 13, 14, 15, 16, 17, 18, 19, 20, 21, 22, 23, 24, or 25 μm in thickness.

In some instances, the biological sample is dried after placement onto the first substrate. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8):431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are detected using immunofluorescence protocols. For example, the sample can be rehydrated, blocked, and permeabilized (3X SSC, 2% BSA, 0.1% Triton X, 1 U/μl RNAse inhibitor for 10 minutes at 4° C.) before being stained with fluorescent primary antibodies (1:100 in 3XSSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

### (ii) Preparation of Biological Sample for Analyte Capture

In some instances (e.g., in an FFPE sample), the biological sample is deparaffinized. Deparaffinization can be achieved using any method known in the art. For example, in some instances, the biological samples is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HC1 for about 1 minute). After the decrosslinking step, the biological sample can be washed (e.g., with 1x PBST).

In some instances, the methods of preparing a biological sample for analyte capture include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

(iii) Simultaneous Capture on the First Substrate and Second Substrate

In some instances, the methods of preparing a biological sample for analyte capture include permeabilizing the sample. In some instances, the biological sample is permeabilized using a phosphate buffer. In some instances, the phosphate buffer is PBS (e.g., 1x PBS). In some instances, the phosphate buffer is PBST (e.g., 1x PBST). In some instances, the permeabilization step is performed multiple times (e.g., 3 times at 5 minutes each).

In some instances, a permeabilization buffer (e.g., any permeabilization buffer described herein) is added to the biological sample. Permeabilization solutions can include, by way of example only, enzymes (e.g., proteinase K, pepsin, and collagenase), detergents (e.g., N-lauroylsarcosine, sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. The permeabilization buffer releases the analyte from the sample, allowing it to diffuse from the sample. In some instances, the biological sample can be treated with a proteinase. In some instances, the proteinase is proteinase K.

In some embodiments, permeabilization occurs using a protease. In some embodiments, the protease is an endopeptidase. Endopeptidases that can be used include but are not limited to trypsin, chymotrypsin, elastase, thermolysis, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, the biological sample is permeabilized prior to capture of the analytes on either the first substrate or the second substrate (or both).

In some instances, the permeabilization step includes application of a permeabilization buffer to the biological sample. In some instances, the permeabilization buffer includes a buffer (e.g., Tris pH 7.5), MgCl2, sarkosyl detergent (e.g., sodium lauroyl sarcosinate), enzyme (e.g., proteinase K, and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 40 minutes.

In some embodiments, the methods provided herein include a permeabilizing step in order to release the analyte. In some embodiments, permeabilization occurs using a protease. In some embodiments, the protease is an endopeptidase. Endopeptidases that can be used include but are not limited to trypsin, chymotrypsin, elastase, thermolysis, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, methods provided herein include permeabilization of the biological sample such that the capture probe can more easily bind to the analyte (i.e., compared to no permeabilization).

In some instances, the permeabilization step includes application of a permeabilization buffer to the biological sample. In some instances, the permeabilization buffer includes a buffer (e.g., Tris pH 7.5), MgCl₂, sarkosyl detergent (e.g., sodium lauroyl sarcosinate), enzyme (e.g., proteinase K), and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 40 minutes.

In some embodiments, the analyte is released using an endoribonuclease. In some embodiments, the endoribonuclease is an RNAse. The RNase can be RNase H, RNase A, RNase C, or RNase I. In some embodiments, the RNase H is RNase H1, RNase H2, or RNase H1, or RNase H2.

In some instances, the releasing step is performed using a releasing buffer. In some instances, the release buffer includes one or more of a buffer (e.g., Tris pH 7.5), enzyme (e.g., RNAse H) and nuclease-free water. In some instances, the releasing step is performed at 37° C. In some instances, the releasing step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 30 minutes.

In some instances, the releasing step occurs after the permeabilization step. In some instances, the releasing step occurs at the same time as the permeabilization step (e.g., in the same buffer).

In some embodiments, the reagent medium (e.g., the permeabilization buffer) comprises one or more of sodium dodecyl sulfate (SDS), proteinase K, pepsin, N-lauroylsarcosine, RNAse, and a sodium salt thereof.

In some instances, a hydrogel is used to enhance spatial resolution. In some embodiments, a biological sample (e.g., tissue section) is embedded in a hydrogel. In some embodiments, hydrogel subunits are infused into the biological sample, and polymerization of the hydrogel is initiated by an external or internal stimulus. A “hydrogel” as described herein can include a cross-linked 3D network of hydrophilic polymer chains. A “hydrogel subunit” can be a hydrophilic monomer, a molecular precursor, or a polymer that can be polymerized (e.g., cross-linked) to form a three-dimensional (3D) hydrogel network. Additional disclosure of hydrogels is found in WO 2020/176788 and U.S. Patent Application Publication No. 2020/0277663, each of which is incorporated by reference in its entirety.

In some instances, analytes migrate through the biological sample. In some instances, analytes migrate from the biological sample to the first and/or second probe. In some instances, migration disclosed herein is passive migration. As some migration is passive, analytes (e.g., mRNA) can migrate in any direction. In some instances, probes on the first substrate capture analytes that migrate passively. In some instances, probes on the second substrate capture analytes that migrate passively.

In some embodiments, after a certain period of time (e.g., about 5 minutes to about 10 hours, about 5 minutes to about 5 hours, about 5 minutes to about 1 hour, about 5 minutes to about 45 minutes, about 5 minutes to about 30 minutes, about 5 minutes to about 15 minutes, about 15 minutes to about 10 hours, about 15 minutes to about 5 hours, about 15 minutes to about 1 hour, about 15 minutes to about 45 minutes, about 15 minutes to about 30 minutes, about 30 minutes to about 10 hours, about 30 minutes to about 5 hours, about 30 minutes to about 1 hour, about 30 minutes to about 45 minutes, about 45 minutes to about 10 hours, about 45 minutes to about 5 hours, about 45 minutes to about 1 hour, about 1 hour to about 10 hours, about 1 hour to about 5 hours, about 1 hour to about 1.5 hours, about 1.5 hours to about 10 hours, about 1.5 hours to about 5 hours, about 1.5 hours to about 2 hours, about 2 hours to about 10 hours, about 2 hours to about 5 hours, about 2 hours to about 3 hours, about 2.5 hours to about 10 hours, about 2.5 hours to about 5 hours, about 2.5 hours to about 3 hours, about 3 hours to about 10 hours, about 3 hours to about 5 hours, about 4 hours to about 10 hours, about 4 hours to about 5 hours, about 5 hours to about 10 hours, about 6 hours to about 10 hours, about 7 hours to about 10 hours, about 8 hours to about 10 hours, or about 9 hours to about 10 hours), one or more analytes have passively migrated and been captured by the capture probe(s) on the first substrate. In some embodiments, after a certain period of time as previously listed, one or more analytes have passively migrated and been captured by the capture probe(s) on the second substrate. In some instances, at least about 80%, or at least about 90% of all analytes are captured by probes on the second substrate. It is contemplated that while a portion of the analytes, for example those in close proximity to the first substrate capture probes, will passively migrate and be captured on the first substrate, whereas the majority of the analytes, those that are not in close proximity to the first substrate, will passively migrate to the second substrate and be captured by the barcoded capture probes.

In some embodiments, there is a gap (e.g., a space) between the first substrate/biological sample and the second substrate that is filled with one or more solutions. In some embodiments, the one or more solutions between the first substrate/biological sample and the second substrate can include a permeabilization buffer (e.g., any of the permeabilization buffers described herein). In some embodiments, the one or more solutions can include a buffer that can maintain the pH at a relatively constant value. In some instances, the gap between the biological sample and the second substrate with the second set of capture probes is about 0.5 μm, about 1.0 μm, about 1.5 μm, about 2.0 μm, about 2.5 μm, about 3.0 μm, about 3.5 μm, about 4.0 μm, about 4.5 μm, about 5.0 μm, or more. In some instances, the distance between the biological sample and the second set of capture probes is about 2.5 μm.

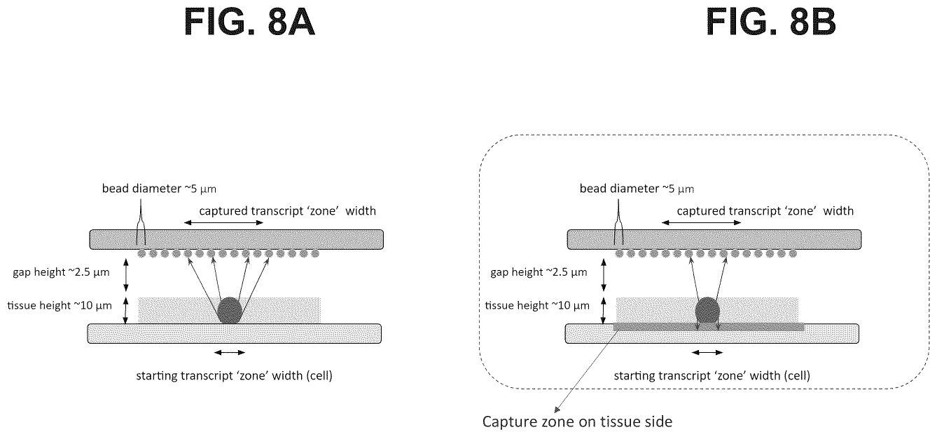

Analytes migrate passively and are therefore free to migrate in any direction. Ultimately, it is contemplated that the analytes are captured by a probe, with a higher number of analytes captured by probes most adjacent to the starting position of the analyte. To decrease broad diffusion of the analytes to probes on the second substrate, it is contemplated that the probes on the first substrate will capture analytes that diffuse along the plane of the first substrate (i.e., through the tissue to the proximate capture probes on the first substrate). Thus, some analytes are captured by the first substrate (and are not typically analyzed to identify the spatial location and sequence of the analyte). However, in some instances, the analytes captured by the first substrate would have ultimately been captured by the probes on the second substrate that are more distal to the original position, creating a “captured analyte zone” as shown in FIG. 8A that is wider than if the probes on the first substrate were not present (FIG. 8B). In some instances, the captured analyte zone is a captured transcript zone. That is, transcripts (i.e., mRNA) are captured in this area of the first substrate.

In some instances, compared to a sandwich configuration that does not include probes on the first substrate, the spatial resolution of analytes from a biological sample is increased. Thus, in some instances, the resolution of detection of analytes is increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 1.5 fold, about 2-fold, about 3-fold, about 4-fold, about 5-fold, about 10-fold, about 15-fold, about 20-fold, or more compared to a sandwich configuration that does not include probes on the first substrate.

In some instances, the resolution of detection of an analyte can be determined by measuring the width of the captured analyte zone. In some instances, increased resolution of detection of the analyte corresponds to decreased width of the captured analyte zone. In some instances, the width of the captured analyte zone is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more compared to a sandwich configuration that does not include probes on the first substrate.

### (iv) Analyte Analysis on the First Substrate

As disclosed herein, analytes are captured on first substrate. As shown in FIG. 7, a biological sample 704 comprises a plurality of analytes that migrate passively from the biological sample. That is, in some instances, the plurality of analytes migrates towards the first substrate 702. In some instances, the plurality of analytes also migrates to the second substrate 706. The first substrate 702 can be used for a variety of functions, including but not limited to examination of captured of expression of an analyte (e.g., an mRNA that hybridizes to a probe) or any other analyte in the biological sample (e.g., a protein, a second mRNA). In some instances, analysis of the analytes captured on the first substrate 702 includes in situ detection of the analyte. For instance, in situ hybridization for captured analyte can be performed on the first substrate. In some instance, the in situ hybridization is fluorescent in situ hybridization.

In some instances, the probes arranged on a lawn on the first substrate can be used to image a sample using methods disclosed herein. In some instances, after an analyte is captured by the probe on the first substrate in a sandwich conformation, the first substrate is removed from the conformation and the location of the analyte is detected. In some embodiments, after interaction of the analyte with the capture probe on the first substrate, one or more target-specific reactions are performed in the biological sample. Examples of target-specific reactions include, but are not limited to, ligation of target specific adaptors, probes and/or other oligonucleotides, target specific amplification using primers specific to one or more analytes, and target-specific detection using in situ hybridization (e.g. fluorescent in situ hybridization (FISH)), DNA microscopy, and/or antibody detection. In some embodiments, a capture probe includes capture domains targeted to target-specific products (e.g., amplification or ligation). In some instances, the methods provide a continuous tissue optimized-like image in addition to the gene expression data from the same tissue section, which can be used for quantifying performance, provide additional information for the gene expression assay. In some instances, fluorescent nucleotides are used to detect an analyte of interest. For example, in some instances, labeled oligonucleotides can be detected by a probe on the first substrate and imaged.

In some embodiments, a probe on the first substrate captures one or more analytes. In some embodiments, the location of the one or more analytes captured on the first substrate are determined by immunofluorescence. In some embodiments, one or more detectable labels (e.g., fluorophore-labeled antibodies) bind to the one or more analytes that are captured (hybridized to) by a probe on the first substrate and the location of the one or more analytes is determined by detecting the labels under suitable conditions. In some embodiments, one or more fluorophore-labeled antibodies are used to conjugate to a moiety that associates with a probe on the first substrate or the analyte that is hybridized to the probe on the first substrate. In some instances, the location(s) of the one or more analytes is determined by imaging the fluorophore-labeled antibodies when the fluorophores are excited by a light of a suitable wavelength. In some embodiments, the location of the one or more analytes in the biological sample is determined by correlating the immunofluorescence data to an image of the biological sample.

In some instances, referring to FIG. 7, the captured analyte on the first substrate 702 is amplified using in situ amplification methods. For instance, in some embodiments, a second strand synthesis can be performed to create a complimentary copy of the analyte (i.e., using the analyte as a template). In some instances, amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction. Amplification methods are further disclosed in WO 2020/176788 and U.S. Patent Application Publication No. 2020/0277663, each of which is incorporated by reference in its entirety.

In some embodiments, in situ amplification is performed by rolling circle amplification. In some embodiments, the capture probe to be amplified includes sequences (e.g., docking sequences, functional sequences, and/or primer sequences) that enable rolling circle amplification. In one example, referring to FIG. 7, one or more probes on the first substrate 702 can include a functional sequence that is capable of binding to a primer used for amplification. In another example, one or more probes on the first substrate 702 can include one or more docking sequences (e.g., a first docking sequence and a second docking sequence) that can hybridize to one or more oligonucleotides (e.g., a padlock probe(s)) used for rolling circle amplification. In some embodiments, additional probes are affixed to the first substrate 702, where the additional probes include sequences (e.g., a docking sequence(s), a functional sequence(s), and/or a primer sequence(s)) that enable rolling circle amplification. In some embodiments, the spatial array is contacted with an oligonucleotide (e.g., a padlock probe). As used herein, a “padlock probe” refers to an oligonucleotide that has, at its 5′ and 3′ ends, sequences that are complementary to adjacent or nearby target sequences (e.g., docking sequences) on a capture probe. Upon hybridization to the target sequences (e.g., docking sequences), the two ends of the padlock probe are either brought into contact or an end is extended until the two ends are brought into contact, allowing circularization of the padlock probe by ligation (e.g., ligation using any of the methods described herein). In some embodiments, after circularization of the oligonucleotide, rolling circle amplification can be used to amplify the ligation product, which includes at least a capture domain and a spatial barcode from the capture probe. In some embodiments, amplification of the capture probe using a padlock oligonucleotide and rolling circle amplification increases the number of capture domains and the number of spatial barcodes on the spatial array.

After capture and optional amplification of the analyte on the first substrate 702, in some instances, the analyte is detected. In some instances, capture probes hybridize to the analyte or an analyte derivative (e.g., an amplified product e.g., from rolling circle amplification).

### (v) Analyte Analysis on the Second Substrate

In some embodiments, as disclosed herein, the analyte hybridizes to the capture probe on the second substrate. Additionally, referring to FIG. 7, the analytes migrate to the second substrate 706 and are captured by the second capture probes (e.g., 707). After capture of the analytes on the second substrate, in some instances, the methods provided herein include determining the spatial expression (e.g., abundance and location) of the analytes in the biological sample using the captured analytes on the second substrate. For example, the analytes may be captured by the second capture probes in a manner that retains spatial context of the analytes in the biological sample. Generally, after hybridization of the analyte to the capture probe on the second substrate, the capture probe is extended at the 3′ end and a copy of the additional capture probe components (e.g., the spatial barcode) of the capture probe is synthesized. In some embodiments, reverse transcription (RT) reagents can be added to the permeabilized biological samples. Incubation with the RT reagents can produce spatially-barcoded full-length cDNA from the captured analytes (e.g., polyadenylated mRNA). Second strand reagents (e.g., second strand primers, enzymes) can additionally be added to initiate second strand synthesis.

In some embodiments, one or more analytes migrate through a solution and specifically bind (e.g., hybridize) to the capture domains on the capture probes of the second substrate. In some embodiments, one or more mRNAs migrate through a solution that includes a permeabilization buffer and bind (e.g., hybridize) to the capture domains on the capture probes on the second substrate. In some embodiments, the sequence of all or a portion of the capture probes (e.g., the spatial barcode or a portion thereof) or a complement thereof, on the second substrate and all or a portion of the sequence of the corresponding captured analyte (or a complement thereof) are determined. In some embodiments, the location of the one or more analytes in the biological sample are determined based on all or a portion of the sequence of the capture probes (e.g., the spatial barcode or a portion thereof) on the array, or a complement thereof, and all or a portion of the sequence of the corresponding captured one or more analytes, or a complement thereof. In some embodiments, determining the location of the one or more analytes in the biological sample includes determining all or a portion of the sequence of the capture probes (e.g., the spatial barcode, or a portion thereof) on the array, or a complement thereof, and all or a portion of the sequence of the corresponding captured one or more analytes, or complement thereof, and correlating such sequence information to an image of the biological sample. Some embodiments of these methods further include obtaining an image of the biological sample.

In some embodiments, after an analyte from the sample has hybridized or otherwise been associated with a capture probe on the second substrate according to any of the methods described above in connection with the general spatial cell-based analytical methodology, the barcoded constructs that result from hybridization/association are analyzed.

In some embodiments, after contacting a biological sample with a second substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with a second substrate comprising a plurality of capture probes, wherein a capture probe of the plurality captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain) In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, provided herein are methods for spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with a second substrate comprising a plurality of capture probes, wherein a capture probe of the plurality captures the biological analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting at least a portion of the biological sample to spatial omics analysis (e.g., spatial transcriptomic analysis). In some embodiments, the method further includes subjecting a region of interest in the biological sample to spatial omics analysis (e.g., spatial transcriptomic analysis). In some embodiments, one or more of the capture probes includes a capture domain In some embodiments, one or more of the capture probes comprises a unique molecular identifier (UMI). In some embodiments, one or more of the capture probes comprises a cleavage domain In some embodiments, the cleavage domain comprises a sequence recognized and cleaved by a uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™ (available from Lucigen, Middleton, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended capture probe.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the analyte and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze a barcoded analyte or derivative thereof. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

(vi) Analyte Analysis on the First or Second Substrate using Templated Ligation

In some instances, instead of capturing the analytes on the first or second substrate, a derivative of an analyte is captured on the first or second substrate. In some instances, the derivative of the analyte is a ligation product that comprises two or more nucleic acid sequences that hybridize to adjacent sequences of the analyte. Once templated ligation is performed, the biological sample can be permeabilized and both subtrates can capture the ligation product (similar to capture of an analyte). Capture of the ligation product is similar to capture of the analyte because the ligation product comprises a poly(A) tail similar to an mRNA analyte. Additional features of templated ligation are now provided.

Templated ligation or RNA-templated ligation (RTL) is a process that includes multiple oligonucleotides (also called “oligonucleotide probes” or simply “probes,” and a pair of probes can be called interchangeably “first probes” and “second probes,” or “first probe oligonucleotides” and “second probe oligonucleotides,”) that hybridize to adjacent complementary analyte (e.g., mRNA) sequences. Upon hybridization, the two oligonucleotides are ligated to one another, creating a ligation product in the event that both oligonucleotides hybridize to their respective complementary sequences. In some instances, at least one of the oligonucleotides includes a sequence (e.g., a poly-adenylation sequence) that can be hybridized to a probe on an array described herein (e.g., the probe comprises a poly-thymine sequence in some instances). In some instances, prior to hybridization of the poly-thymine to the poly(A) sequence, an endonuclease digests the analyte that is hybridized to the ligation product. This step frees the newly formed ligation product to hybridize to a capture probe on a spatial array. In this way, templated ligation provides a method to perform targeted RNA capture on a spatial array.

Targeted RNA capture allows for examination of a subset of RNA analytes from the entire transcriptome. In some embodiments, the subset of analytes includes an individual target RNA. In some instances, the presence of the ligation product that is created as a result of the templated ligation methods described herein indicates that the individual target RNA is present. In some instances, the absence of the ligation product that is created as a result of the templated ligation methods described herein indicates that the individual target RNA is not present. In some instances, an absence of the ligation product is because one of the oligonucleotide probes did not hybridize to the analyte. In some instances, an absence of the ligation product is because both (e.g., two) of the oligonucleotide probes did not hybridize to the analyte.

In some embodiments, the subset of analytes detected using methods disclosed herein includes two or more (e.g., 2, 3, 4, 5, 6, 7, 8, 9, 10, or more) targeted RNAs. In some embodiments, the subset of analytes includes one or more mRNAs transcribed by a single gene. In some embodiments, the subset of analytes includes one or more mRNAs transcribed by more than one targeted genes. In some embodiments, the subset of analytes includes one or more mRNA splice variants of one or more targeted genes. In some embodiments, the subset of analytes includes non-polyadenylated RNAs in a biological sample. In some embodiments, the subset of analytes includes detection of mRNAs having one or more (e.g., 2, 3, 4, 5, or more) single nucleotide polymorphisms (SNPs) in a biological sample.

In some embodiments, the subset of analytes includes mRNAs that mediate expression of a set of genes of interest. For example, in some instances, the subset of analytes detected using the templated ligation methods disclosed herein include analytes that are translated into transcription factors that control one or more cellular pathways. In some embodiments, the subset of analytes includes mRNAs that share identical or substantially similar sequences, which mRNAs are translated into polypeptides having similar functional groups or protein domains. In some embodiments, the subset of analytes includes mRNAs that do not share identical or substantially similar sequences, which mRNAs are translated into proteins that do not share similar functional groups or protein domains. In some embodiments, the subset of analytes includes mRNAs that are translated into proteins that function in the same or similar biological pathways. In some embodiments, the biological pathways are associated with a pathologic disease. For example, targeted RNA capture can detect genes that are overexpressed or underexpressed in a cancer sample.

In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, about 1000, or more analytes.

In some embodiments, the subset of analytes detected by targeted RNA capture methods provided herein includes a large proportion of the transcriptome of one or more cells. For example, the subset of analytes detected by targeted RNA capture methods provided herein can include at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of the mRNAs present in the transcriptome of one or more cells.

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some embodiments, a biological sample (e.g., tissue section) can be fixed with methanol, stained with hematoxylin and eosin, and imaged. In some embodiments, fixing, staining, and imaging occurs before one or more oligonucleotide probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the methods of targeted RNA capture as disclosed herein include hybridization of multiple probe oligonucleotides. In some embodiments, the methods include 2, 3, 4, or more probe oligonucleotides that hybridize to one or more analytes of interest. In some embodiments, the methods include two probe oligonucleotides. In some embodiments, the probe oligonucleotide includes sequences complementary that are complementary or substantially complementary to an analyte. For example, in some embodiments, the probe oligonucleotide includes a sequence that is complementary or substantially complementary to an analyte (e.g., an mRNA of interest (e.g., to a portion of the sequence of an mRNA of interest)). Methods provided herein may be applied to a single nucleic acid molecule or a plurality of nucleic acid molecules. A method of analyzing a sample comprising a nucleic acid molecule may comprise providing a plurality of nucleic acid molecules (e.g., RNA molecules), where each nucleic acid molecule comprises a first target region (e.g., a sequence that is 3′ of a target sequence or a sequence that is 5′ of a target sequence) and a second target region (e.g., a sequence that is 5′ of a target sequence or a sequence that is 3′ of a target sequence), a plurality of first probe oligonucleotides, and a plurality of second probe oligonucleotides.

In some embodiments, the templated ligation methods that allow for targeted RNA capture as provided herein include a first probe oligonucleotide and a second probe oligonucleotide. The first and second probe oligonucleotides each include sequences that are substantially complementary to the sequence of an analyte of interest. By substantially complementary, it is meant that the first and/or second probe oligonucleotide is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in an analyte. In some instances, the first probe oligonucleotide and the second probe oligonucleotide hybridize to adjacent sequences on an analyte.

In some embodiments, the first and/or second probe as disclosed herein includes one of at least two ribonucleic acid bases at the 3′ end; a functional sequence; a phosphorylated nucleotide at the 5′ end; and/or a capture probe binding domain. In some embodiments, the functional sequence is a primer sequence. The capture probe binding domain is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or both. In some embodiments, the capture probe binding domain includes a random sequence (e.g., a random hexamer or octamer). In some embodiments, the capture probe binding domain is complementary to a capture domain in a capture probe that detects a particular target(s) of interest.

In some embodiments, a capture probe binding domain blocking moiety that interacts with the capture probe binding domain is provided. In some instances, the capture probe binding domain blocking moiety includes a nucleic acid sequence. In some instances, the capture probe binding domain blocking moiety is a DNA oligonucleotide. In some instances, the capture probe binding domain blocking moiety is an RNA oligonucleotide. In some embodiments, a capture probe binding domain blocking moiety includes a sequence that is complementary or substantially complementary to a capture probe binding domain In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a ligated probe) to a capture probe. In some embodiments, a capture probe binding domain blocking moiety comprises a poly-uridine sequence, a poly-thymidine sequence, or both.

In some embodiments, the first probe oligonucleotide hybridizes to an analyte. In some embodiments, the second probe oligonucleotide hybridizes to an analyte. In some embodiments, both the first probe oligonucleotide and the second probe oligonucleotide hybridize to an analyte. Hybridization can occur at a target having a sequence that is 100% complementary to the probe oligonucleotide(s). In some embodiments, hybridization can occur at a target having a sequence that is at least (e.g., at least about) 80%, at least (e.g. at least about) 85%, at least (e.g. at least about) 90%, at least (e.g. at least about) 95%, at least (e.g. at least about) 96%, at least (e.g. at least about) 97%, at least (e.g. at least about) 98%, or at least (e.g. at least about) 99% complementary to the probe oligonucleotide(s).

After hybridization of the first and second probe oligonucleotides, in some embodiments, the first probe oligonucleotide is extended. After hybridization, in some embodiments, the second probe oligonucleotide is extended. Extending probes can be accomplished using any method disclosed herein. In some instances, a polymerase (e.g., a DNA polymerase) extends the first and/or second oligonucleotide.

In some embodiments, methods disclosed herein include a wash step. In some instances, the wash step occurs after hybridizing the first and the second probe oligonucleotides. The wash step removes any unbound oligonucleotides and can be performed using any technique or solution disclosed herein or known in the art. In some embodiments, multiple wash steps are performed to remove unbound oligonucleotides.

In some embodiments, after hybridization of probe oligonucleotides (e.g., first and the second probe oligonucleotides) to the analyte, the probe oligonucleotides (e.g., the first probe oligonucleotide and the second probe oligonucleotide) are ligated together, creating a single ligated probe that is complementary to the analyte. Ligation can be performed enzymatically or chemically, as described herein.

### (c) Biological Samples and Analytes

Methods disclosed herein can be performed on any type of sample. In some embodiments, the sample is a solid tissue sample. In some embodiments, the sample is a fresh tissue. In some embodiments, the sample is a frozen sample. In some embodiments, the sample was previously frozen, e.g., is a fresh frozen sample. In some embodiments, the sample is a formalin-fixed, paraffin embedded (FFPE) sample. As used herein, the terms “sample” and “biological sample” are interchangeable.

A “biological sample” is obtained from the subject for analysis using any of a variety of techniques including, but not limited to, biopsy, surgery, and laser capture microscopy (LCM), and generally includes cells and/or other biological material from the subject. In addition to the subjects described above, a biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can also include fetal cells. For example, a procedure such as amniocentesis can be performed to obtain a fetal cell sample from maternal circulation. Sequencing of fetal cells can be used to identify any of a number of genetic disorders, including, e.g., aneuploidy such as Down's syndrome, Edwards syndrome, and Patau syndrome. Further, cell surface features of fetal cells can be used to identify any of a number of disorders or diseases.

Biological samples can also include immune cells. Sequence analysis of the immune repertoire of such cells, including genomic, proteomic, and cell surface features, can provide a wealth of information to facilitate an understanding the status and function of the immune system. By way of example, determining the status (e.g., negative or positive) of minimal residue disease (MRD) in a multiple myeloma (MM) patient following autologous stem cell transplantation is considered a predictor of MRD in the MM patient (see, e.g., U.S. Patent Application Publication No. 2018/0156784, the entire contents of which are incorporated herein by reference).

Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

Cell-free biological samples can include extracellular polynucleotides. Extracellular polynucleotides can be isolated from a bodily sample, e.g., blood, plasma, serum, urine, saliva, mucosal excretions, sputum, stool, and tears.

As discussed above, a biological sample can include a single analyte of interest, or more than one analyte of interest. Methods for performing multiplexed assays to analyze two or more different analytes in a single biological sample is discussed in a subsequent section of this disclosure.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells,

In some instances, the cancer is breast cancer. In some instances, the breast cancer is triple positive breast cancer (TPBC). In some instances, the breast cancer is triple negative breast cancer (TNBC).

In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more analytes are captured. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the substrate.

In some embodiments, the FFPE sample is deparaffinized, permeabilized, equilibrated, and blocked before analyte capture. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

In some instances, the methods disclosed herein include preparation of a biological sample. In some instances, the biological sample is a tissue sample. In some instances, the biological sample is a tissue section. In some instances, the tissue is a fresh sample. In some instances, the fresh sample has been sectioned. In some instances, the tissue is a frozen sample. In some instances, the frozen sample has been sectioned. In some instances, the tissue is a fixed sample. In some instances, the tissue is a formalin-fixed paraffin-embedded (FFPE) tissue, a PFA fixed sample or an acetone fixed sample. Any other suitable tissue samples described herein can also be used in the methods.

In some instances, the biological sample is a live sample. In some instances, the biological sample is a section of a live tissue sample. In some instances, the biological sample is a culture of cells (e.g., as disclosed herein). Live sample as used herein refers to a sample that maintains at least 60%, 65%, 70%, 75%, 80%, 85%, 90%, 91%, 92%, 93%, 94%, 95%, 96%, 97%, 98%, 99% or 100% viability. In some instances, the live tissue is sectioned using a vibratome.

The apparatus, systems, methods, and compositions described in this disclosure can be used to detect and analyze a wide variety of different analytes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria).

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis.

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids.

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

Additional examples of analytes include mRNA and cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein). In some embodiments, a perturbation agent can be a small molecule, an antibody, a drug, an aptamer, a miRNA, a physical environmental (e.g., temperature change), or any other known perturbation agents.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

In general, the systems, apparatus, methods, and compositions can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (d) Kits, Systems, and Compositions

In some embodiments, also provided herein are kits, systems, and apparatuses, each configured to perform the methods disclosed herein.

In some instances, the kits and systems include one or more reagents to detect one or more analytes described herein. In some instances, the kits and systems include a first substrate comprising a plurality of capture probes, each of which includes a capture domain (e.g., a poly(T) sequence. In some instances, the kits and systems include a second substrate comprising a plurality of capture probes, each of which includes a capture domain (e.g., a poly(T) sequence and a spatial barcode. It is appreciated that any of the capture probes disclosed herein can be designed so that a user can detect any analyte of interest.

A non-limiting example of a kit used to perform any of the methods described herein includes: (a) a first substrate comprising a plurality of first capture probes, wherein a first capture probe of the plurality of first capture probes comprises a first capture domain; (b) a second substrate comprising a plurality of second capture probes, wherein a second capture probe of the plurality of second capture probes comprises (i) a spatial barcode and (ii) a second capture domain; and (c) instructions for performing any of the methods disclosed herein.

A non-limiting example of a kit used to perform any of the methods described herein includes: (a) a first substrate comprising a plurality of first capture probes, wherein a first capture probe of the plurality of first capture probes comprises a first capture domain; (b) a second substrate comprising a plurality of second capture probes, wherein a second capture probe of the plurality of second capture probes comprises (i) a spatial barcode and (ii) a second capture domain; and (c) the biological sample.

It is further appreciated that the kits, systems, and apparatuses can include one or more reagents necessary to perform any of the methods disclosed herein. In addition, the kits, systems, and apparatuses can also include one or more enzymes (e.g., polymerase; reverse transcriptase; ligase) necessary to perform any of the methods disclosed herein.

### EXAMPLES

### Example 1—Enhancing Spatial Resolution of an Analyte on a Spatial Array

This example provides an exemplary method for enhancing the resolution of an analyte in a biological sample.

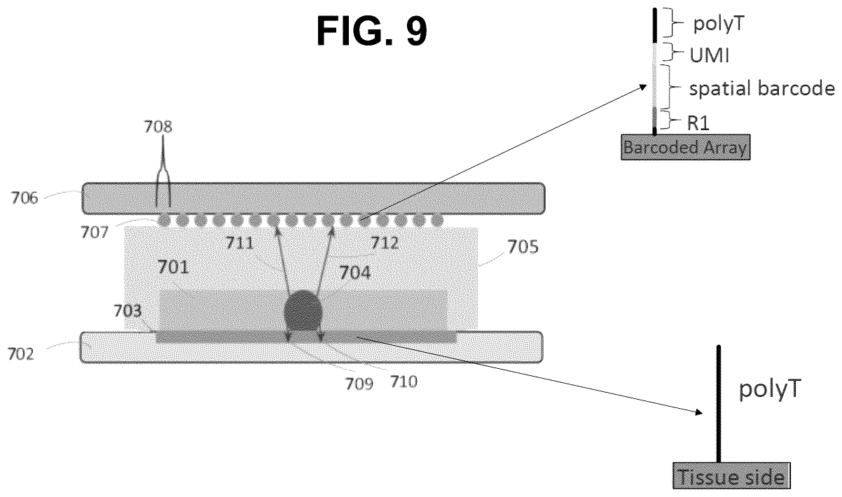

In a non-limiting example, and as shown in FIG. 8B, a 10 μm tissue sample is sectioned and placed on a first substrate. The first substrate includes a lawn of capture probes, each of which includes a capture domain (e.g., a poly-thymine sequence). After placing the biological sample on the first substrate, a second substrate is placed on top, superior to, the first substrate, creating a sandwich configuration with the biological sample between the first substrate and the second substrate. The second substrate includes a plurality of capture probes, each of which includes both a capture domain (e.g., a poly-thymine sequence) and a spatial domain Exemplary probe arrangements the first substrate (e.g., the “Tissue Slide”) and for the second substrate (e.g., the “Barcoded Array”) are shown in FIG. 9. A permeabilization buffer comprising proteinase K is added to the biological sample, allowing the analytes to migrate from the biological sample.

Analytes migrate passively in all directions and are captured by capture probes each of the two substrates (e.g., by hybridization of the poly(A) tail of an analyte to the poly(T) sequence of the capture probe. Analytes that migrate along the plane of the first substrate are captured by capture probes on the first substrate, disallowing them to “fan” out to capture probes on the second substrate. The net result is that analytes that are captured by the second substrate are captured in an area with a narrower width of migration compared to a sandwich type substrate configuration that does not include probes on the first substrate (shown in FIG. 8A).

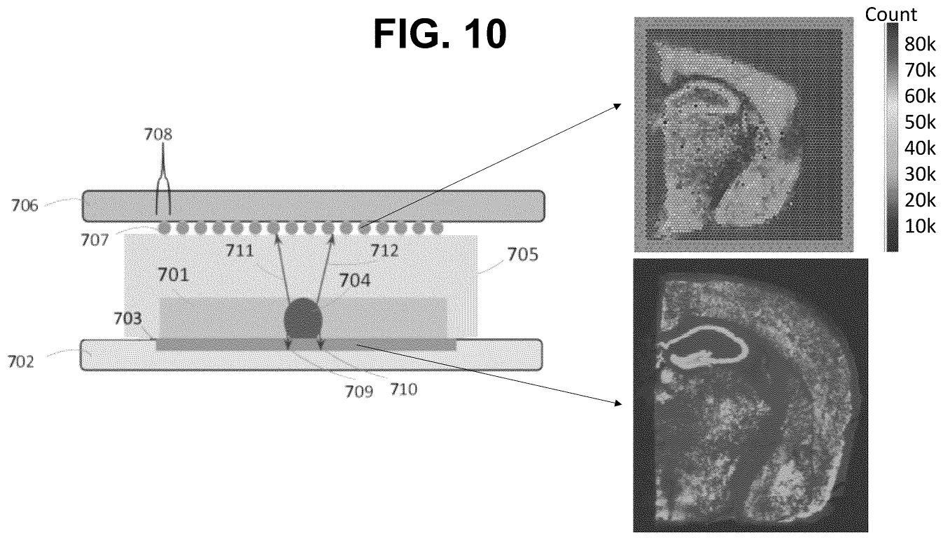

Analytes that are captured on the first substrate are analyzed for detection of the analytes. For instance, once analytes are captured by the capture probes on the first substrate, the sandwich configuration can be separated, and in situ analysis can be performed on the analytes captured on the first substrate. As an exemplary in situ method, and without being bound by theory, fluorescent probes are designed to contain sequences complementary to the captured analyte on the first substrate. After a brief incubation period of the fluorescent probes, analytes can be detected based on hybridization and fluorescent readout of the hybridized fluorescent probes using a fluorescent microscope. See e.g., FIG. 10, bottom right image.

Analytes that are captured on the second substrate prepared for analysis. In brief, the captured analytes on the second substrate are immobilized on the second substrate via the poly(A) tail. The captured analytes are copied, using the analyte as a template; and the extension product is released from the spatial array. Briefly, the tissues are incubated with a second strand extension mix comprising DNA polymerase for 25 minutes at 53° C. Following incubation, the second strand extension mix is removed from the tissues and the tissues are washed with 2X SSC. A solution of KOH is added to each of the tissue wells at room temperature for 10 minutes to release the extension product from the spatial array and the supernatant from each tissue well is transferred for quantification, and library preparation. Sample quantification is performed using qPCR and KAPA SYBR® FAST qPCR master mix according to the manufacturer's instructions. For library preparation, samples are indexed using an Amp Mix that included dual indexing primers and an Amp Mix. Nucleic acids from the amplification reaction are then sequenced and analyzed. An image of the sample is generated based on analyte detection. See e.g., FIG. 10, top right image.

### Example 2: Efficient Analyte Capture from Slide-Mounted Fresh Frozen Mouse Brain Sections onto Spatial Array Slides

Analyte capture onto spatially barcoded arrays and subsequent sequencing was demonstrated under sandwich and non-sandwich conditions. For the test (sandwiching) condition, archived tissue-mounted standard glass slides containing hematoxylin/eosin stained fresh frozen mouse brain sections were used. For control (non-sandwich) condition, GEx array slides with hematoxylin/eosin stained fresh frozen mouse brain sections mounted directly onto the array area were used. Under both conditions, tissue sections were subjected to a hematoxylin destaining step. Slides processed according to the “sandwiching” condition were briefly dried at 37° C., then mounted in an instrument along with a GEx slide and a permeabilization buffer comprising sarkosyl and proteinase K. Upon sandwich closure in the instrument, the tissue sections were permeabilized for 1 minute. For the tissue-mounted GEx slides processed according to the non-sandwich condition, sections were permeabilized for 5 minutes using the same permeabilization buffer without sandwiching. For both conditions, following permeabilization, captured polyA-containing mRNA transcripts on the GEx slides were reverse transcribed into cDNA, followed by standard sequencing library preparation and sequencing.

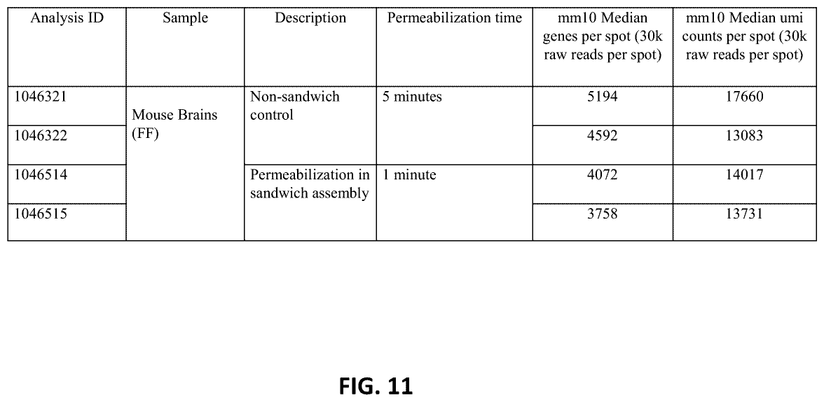

Results depicting median genes per spot and median UMI counts per spot are shown in FIG. 11.

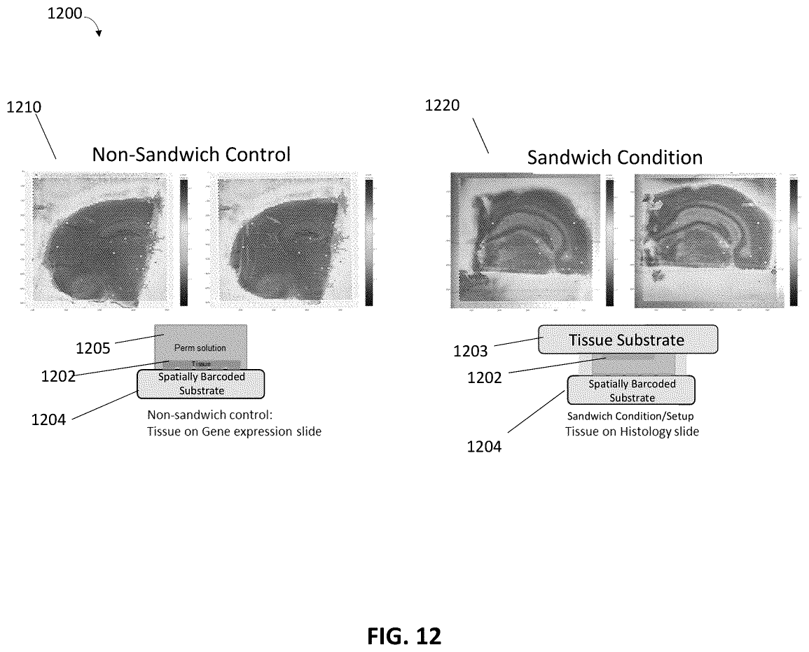

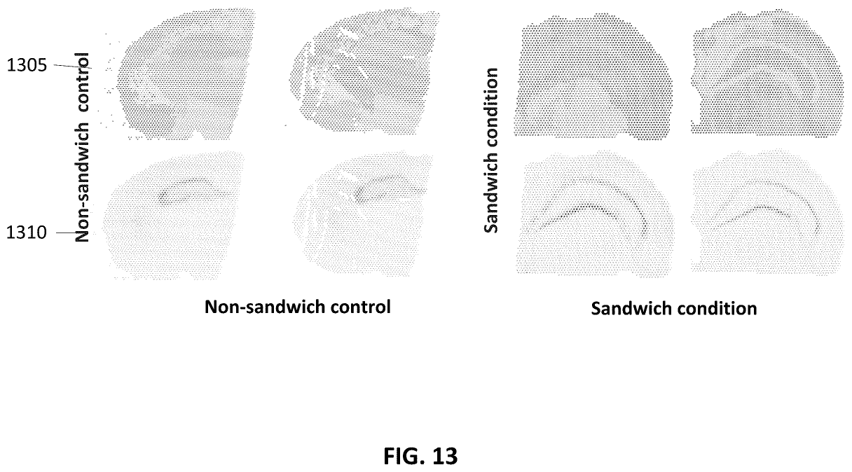

Visual heat map results showing Log10 UMIs are shown in FIG. 12. Spatial patterns of the Log10 UMI counts were similar across the sandwich and non-sandwich conditions. Spatial clustering analysis (top row 1305) and analysis of hippocampal transcript Hpca (bottom row 1310) are depicted in FIG. 13. Spatial patterns were comparable across the sandwich and non-sandwich conditions.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A kit comprising:
(a) a first substrate comprising a plurality of first capture probes, wherein a first capture probe of the plurality of first capture probes comprises a first capture domain;
(b) a second substrate comprising a plurality of second capture probes, wherein a second capture probe of the plurality of second capture probes comprises (i) a spatial barcode and (ii) a second capture domain; and
(c) instructions for performing a method for determining the location of an analyte in a biological sample, the method comprising:
providing the first substrate;
providing the second substrate on the opposite side of the first substrate relative to the biological sample, thereby sandwiching the first substrate, the biological sample, and the second substrate;
hybridizing the analyte to the second capture domain and hybridizing a second analyte to the first capture domain; and
determining sequences of (i) the spatial barcode, or a complement thereof, and (ii) all or a portion of the analyte captured on the second capture domain, or a complement thereof, and using the sequences of (i) and (ii) to determine the location of the analyte in the biological sample.

2. A system for determining the abundance and location of an analyte in a biological sample, the system comprising:
(a) a first substrate comprising a plurality of first capture probes, wherein a first capture probe of the plurality of first capture probes comprises a first capture domain;
(b) a second substrate comprising a plurality of second capture probes, wherein a second capture probe of the plurality of second capture probes comprises (i) a spatial barcode and (ii) a second capture domain;
(c) a biological sample holder comprising: (i) a first member comprising a first retaining mechanism configured to receive the first substrate, (ii) a second member configured to receive the second substrate, and (iii) an alignment mechanism that is connected to at least one of the first member and second member and configured to align the first substrate and the second substrate; and
(d) the biological sample.
